(19) United States
(12) Patent Application Publication
(10) Pub. No.: US 2014/0030365 A1
(43) Pub. Date: Jan. 30, 2014

(54) COMPOSITIONS FOR THE DEBRIDEMENT, GRANULATION AND REEPITHELIALIZATION OF WOUNDS IN MAN

(71) Applicants: Victor Hugo Angulo Borja, Viche (EC); Nuria Sulayne Angulo Conforme, Viche (EC); Victor Graciel Angulo Conforme, Viche (EC)

(72) Inventors: Victor Hugo Angulo Borja, Viche (EC); Nuria Sulayne Angulo Conforme, Viche (EC); Victor Graciel Angulo Conforme, Viche (EC)

(21) Appl. No.: 13/982,717

(22) PCT Filed: Jan. 30, 2012
(86) PCT No.: PCT/IB12/00801

§ 371 Date: Oct. 18, 2013

(30) Foreign Application Priority Data
Jan. 31, 2011 (IT) RM2011A000042

(51) Int. Cl. A61K 36/81; A61K 36/41; A61K 36/21; A61K 36/68

(52) U.S. Cl. CPC A61K 36/81; A61K 36/68; A61K 36/41; A61K 36/21
USPC 424/738

(57) ABSTRACT

Compositions for topical application based on natural products, specifically dried pulverized plants suspended in bees honey, in which the ingredients are combined in proportions predetermined to provide three different types of preparations to be applied at various stages of repair and regeneration of injured tissues. The application of the compositions shortens the stages of healing in the treatment of acute and chronic injuries of any degree of depth and breadth, and of any etiology.

The properties of the preparations include an hemorheological and systemic vascular activity which extends for a certain period even after the end of topical treatment.

### FIELD OF THE INVENTION

The present invention concerns topical compositions for the debridement, granulation and reepithelializaton of wounds in man. More particularly, this invention concerns compositions based on natural products, specifically plants and honey, combined in suitable proportions to form three different types of preparations for topical administration. Such three different types of preparations are suitable to shorten the phases of tissue regeneration in the treatment of acute and chronic wounds of any depth and extension, and of any etiology.

### BACKGROUND OF THE INVENTION

As it is known, wound healing is a complex biological phenomenon aimed at filling the solution of continuity represented by the wound with a definitive structure of connective nature, the scar. Under normal conditions, the healing process is a series of events aimed to the new formation of a tissue having a function to replenish the loss of substance due to the wound. This process occurs through distinct phases, which partially overlap at different times. The methods of healing are partly different depending on whether the injuries were from a cut, such as those surgical, whose margins are sharp and which are sutured juxtaposing the edges of the wound with a minimum loss of substance, or wounds which are left open due to trauma with lacerations, or loss of tissue due to infection or burns, and that, in order to be remedied, they require the formation of a larger amount of tissue filler called “granulation”, with longer times and higher risks of imperfections in the resulting scar. A third mode of healing is then the one that affects the wounds that have met post-operative complications, which require the re-opening of the wound and delicate treatments for cleansing, removing of damaged or infected areas (which is indicated in Italian by the French term “debridement”, i.e. removing necrotic matter in order to prepare the wound healing) and draining.

The process of wound repair typical of the first of the above mention modes of healing, i.e. the case of injuries by cutting sutured and substantially without loss of substance, occurs first of all through an hemostatic phase, wherein coagulation tissue factors are activated and a clot is formed, consisting of a fibrin network in which the corpuscular elements of the blood located in the wound remain imprisoned. Then an inflammatory phase takes place, being the typical response of the organism against pathogens, in which possible microbial agents, foreign bodies and necrotic cells are circumscribed and eliminated by neutrophil granulocytes and by macrophages, which together provide for the cleansing and debridement of wound. After the initial vasoconstriction which occurs in the hemostatic phase, the inflammatory reaction is instead accompanied with a vasodilation, which makes the wound edematous and strongly reddened. As already noted in relation to the partial overlapping of the various phases of the process described here, the inflammatory reaction is also prolonged in part during the subsequent proliferative phase.

The proliferative phase is directed to replace the clot with a solid, final structure, and starts with cell proliferation of the epithelial structures, endothelial cells and connective tissue from the margins of the wound resulting in a tissue having a granular characteristic appearance, called granulation tissue. Neovascularization takes place through the formation of cell outlines at the wound edges which, according to the scaffold formed by the fibrin network, proceed toward the center until joining with those from the other side and constituting a new vascular network (angiogenesis). At the same time the cells proliferation of the epithelium basal layer begins, again starting from the wound edges, and from the connective tissue the fibroblasts proliferation starts. The latter are cell elements that, by secreting hyaluronic acid, give rise to the formation of collagen fibers that will serve to fill the wound.

In the healing processes of not sutured wounds, wherein a loss of substance occurred, the granulation tissue fills the wound starting from the deepest layers, and the lack of an adequate support at this stage not only prolongs the healing process, but also often provokes the irregular filling of the wound, with imperfections and hypertrophy of the resulting scar. The completion of the outer epithelial layer ends the proliferative phase.

In the later step of maturation and remodeling, the wound contraction continues, which began already in the previous step, and goes on even after the complete epithelialization. In the meantime the maturation takes place, as well as the rearrangement and crosslinking of collagen fibers, a process whose duration is extremely variable, and determines final consolidation of the wound area and a gradual normalization of the color of the scar. The remodeling phase can last for months or years, depending on the type of wound and on other factors that may influence the healing process.

Some local factors of the wound may indeed affect the course of wound healing, such as the intensity of the trauma, the presence of hematoma or serous collections, local infection or disease, the presence of foreign bodies or splinters, or alterations in the blood flow. Among the general or systemic factors that may affect the outcomes it is necessary to consider the age and general health of the subject, his nutritional status and possible vitamin deficiencies, the presence of some systemic diseases, especially diabetes, and the simultaneous application of certain therapies, especially the cytostatic therapies and those based on corticoids, which adversely affect the healing process.

The epithelial repair, on the other hand, appears to be influenced by other factors: it is slowed by hypothermia and accelerated by a moderate hyperthermia (up to 40° C.). The significance of this fact is that an increase in the local temperature at the level of the scar, stimulates wound healing, probably by increasing of blood circulation.

It is now widely accepted that the best treatment for a normal surgical injury, is to let it evolve by itself. Only a cleaning is required, i.e., washing with physiological serum and rest, which means avoiding any kind of relaxation of the wound and adjacent skin.

In the past, and to tate, the plants have played an important role as remedies and aids in the treatment of human diseases, particularly in the treatment of wounds plants of all kinds and their derivatives have been part of the therapeutic arsenal, and among these: Agrimonia eupatoria (agrimony), Arctium lappa (burdock), Capsella bursa-pastoris (sheperd's purse), Cupressus sempervivens (mediterranean cypress), Equisetum arvense (puzzlegrass or horsetail), Symphytum officinalis (comfrey), Echinacea spp. (coneflower), Rhammus frangula (frangula), Fucus vesiculosus (bladder wrack), Arbutus uva-ursi (bear grapes), Gentiana lutea (great yellow gentian), Hedera helix (ivy), Citrus×limon (lemon), Plantago major (plantain greater), Zea mays (mais), Malva sylvestris (mallow), Matricaria chamomilla (camomile), Origanum majorana (marjoram), Melilotus officinalis (melilot), Melissa officinalis (lemon balm), Juglans regia (walnut), Carica papaya (papaia), Glycyrrhiza glabra (liquorice), Rosa gallica (rose), Salvia officinalis (sage) e Sambucus nigra (elder). Among the currently most used medicinal plants are mentioned: Aloe vera o Aloe barbadensis (aloe), Calendula officinalis (calendula), Uncaria tormentosa (cat's claw) e Hydrocotyle asiatica (centella asiatica).

It is shown that many extracts topically applied exert a positive influence on wound healing, promoting increased levels of endogenous antioxidant enzymatic and non enzymatic, and promoting angiogenesis. An example worth mentioning is the extract of Hippophae rhamnoides I. turkestanica subspecies (sea buckthorn), a shrub native to Europe and Asia. The plant has been widely used in traditional oriental system of medicine for the treatment of asthma, skin diseases, gastric and lung disorders. All parts of the plant shall be deemed to a source of a large number of bioactive substances such as flavonoids (isorhamnetin, quercetin, myricetin, kaempferol and their glucosidic derivatives), carotenoids (beta-carotene, lycopene), vitamins (C, E, K), tannins, triterpenes, glycerides of palmitic, stearic and oleic acids and some essential amino acids. Thanks to the properties of the active compounds present in the plant, for example, experimental studies conducted on female rats with normal and streptozotocin-induced diabetes by means of the extract of Hippophae rhamnoides, in mixtures in ratio 1:7:1 with Aloe vera extract and Curcuma longa (turmeric), showed efficacy in accelerating the healing of wounds, both acute and chronic (A. Gupta et al., A poly-herbal formulation accelerates normal and impaired diabetic wound healing, Department of Biochemical Pharmacology, Defence Insitute of Physiology and Allied Sciences (DPAS), Delhi, India, asheesh_gupta2001@yahho.co.in).

Among the most widely used medicinal plants worldwide, Aloe vera is widely used to treat many diseases, particularly of dermatology and of varying severity, including forms of diabetes. In several experiments on the treatment of wounds, about 90% of those treated with aloe vera gel are resolved with 72 hours earlier than those not treated with this active ingredient. In septic injury, however, recovery is much slower than with conventional treatment.

Another natural product, not directly from plant source, which is used to treat wounds since ancient times is the honey bee. Laboratory tests on animals and in vitro assays indicate that honey can accelerate wound repair due to its action on cell division, on the synthesis and maturation of collagen, the contraction and epithelialization of the wound and on improvement of nutritional. The honey has antibacterial properties, for its high content of hydrogen peroxide, as well as high levels of antioxidants that protect tissues from free radicals. Anti-inflammatory properties have been described, which reduce edema, exudate and local pain. Similarly, its acidity (pH below 4) favors the antibacterial action of macrophages, since an acid pH within the vacuoles facilitates the bacterial lysis, while it reduces the production of toxic ammonium.

It is known that in acute wounds the application of honey can reduce the healing time compared to conventional treatments; on the other hand, it has been also found that in chronic wounds honey, in addition to conventional compression bandage, does not significantly improve the healing varicose leg wound.

In order to successfully address the underlying causes of various chronic or incurable wounds of any kind, devices to restore or at least cover the surfaces of the lesions have been recently developed. Artificial substrates dermal collagen-based are made, glycosaminoglycans, chitosan, or similar materials, in particular known by the name of “scaffold” consisting of porous three-dimensional structures to be used as matrices for the within tissue cells development to regenerate or rebuild. Such substrates promote the regeneration of vascular and also nerve elements, and components of the structure of the sub-dermal connective tissue, to provide with skin grafts from autologous or artificially developed by in vitro culture, the environments that allow them an adequate insertion,

The skin substitutes were found to be a very useful alternative, and sometimes essential in those cases in which the uncovered areas are very extensive, as happens in burns, or when for some particular conditions, autografts cannot be considered as a possibility, for example, when the area from which to obtain the skin is very painful or when there is a risk of developing scars or infection, or when there is a healing too slow. The long-term studies on biopsies taken after years from the sites of application show the regeneration of the elastic fibers and partially of nerve cells, but also in this case, as in the physiological repair of tissues originally healthy, there is no regeneration of the skin attached, such as sebaceous and sweat glands and hair follicles.

Taking into account the persistent infection as a cause for delay or chronic wounds, medications and creams for topical application based on antibiotics were designed and built during the times, with the intention to treat or prevent infections. Among them, the silver-based creams have a global recognition. The use of silver as an antiseptic and disinfectant dates back to ancient times, and was applied to the preparation of medicaments utility universally recognized as the sulfadiazine silver. In the treatment of second-degree burns the silver sulfadiazine is the preferred drug for its anti-inflammatory and stimulating of tissue regeneration action, and also for its analgesic properties.

Iodine is another agent commonly used in antiseptics preparations for the treatment of healing wounds, as well as certain antibiotics for topical use, generally in cream or gel, such as gentamicin, chloramphenicol, neomycin and their combinations.

In addition, alginate based dressings (a chemokine of natural origin) have been proposed, to be used as a vehicle for active molecules to stimulate the regeneration of granulation tissue and accelerate the healing process of wounds. Some examples include alginate hydrogel patch as a vehicle for the factor-1 of stromal regeneration (SDF-1, stromal cell-derived factor-1), which is a factor stimulating tissue regeneration, or in combination with substances that increase the resistance to the infection, as in the ionic dressing alginate/silver carboxymethyl cellulose (SACMC) or also in combination with anti-inflammatory agents. In situations where it is important is to provide moisture, mainly in chronic wounds, the alginate is combined with hydrocolloids, hydrogels, foams or hydrofibres.

As is known, to prepare the wound bed of an acute or chronic wound, of traumatic or infective type, and especially of wounds in which the debris and necrotic material cover substantially the living tissue acting as a barrier which prevents or hinders the penetration and adherence of regenerative substances, a “debridement” of the lesion has to be made first. This consists in removing foreign materials or necrotic tissue, devitalized or infected in the area of the lesion, up to exposure of surrounding healthy tissue, in order to prepare the wound to regeneration. In medical practice use is made of surgical debridement, but in situations where this is hazardous because of the danger of bleeding or other complications, chemical enzymatic debridement, for example by means of collagenase, papain or papain-urea (with or without chlorophyllin) is most useful. In other circumstances a mixt debridement allows even better results.

There is also the alternative practice of using maggots for the debridement of wounds when conventional methods do not yield results, or when there is no contraindication. None of the techniques mentioned have sufficient scientific topic to be better than the others and to be recommend as a technique of choice for special cases.

Taking into account diabetic ulcers, as epidemiologically more common wounds that appear to be difficult to resolve, given the worldwide spread of diabetes diseases, it is known that these lesions consist of the continuity of tissues that can affect, related to its importance and severity, the skin tissue, subcutaneous tissue and finally the bone tissue. The most common location is in the foot, where the lesion may involve the apical regions of the fingers, the joints between the phalanges joints, the heads of the metatarsals (plantar area), heel, the bony prominences of the ankle (tibio-peroneal-mortar talus), and leg. The involvement of the foot is very dangerous for the continuation of the disease, since it often comes to the interest of the subcutaneous tissues and bone, with infections and fistulas that can lead to amputation of the affected regions.

Clinical trial on patients with diabetes shows that in these patients all the metabolic processes are altered. In fact, one of the reasons because a person with diabetes develops serious injury, where a healthy person would have only light scratches, is in the fact that its metabolic control, despite the personal efforts and the medical one, is not the best. This situation is aggravated when the metabolic control is poor, and therefore there is a slowing of the regenerative processes and resistance to infections, and the body fluids of the patient are rather converted in excellent culture media. Under such conditions, saprophytic germs become harmful and opportunistic, fungi become invasive, facultative aerobic bacteria become anaerobic.

In studies that led to the present invention, with the aim of seeking a topical preparation that is effective to shorten the treatment of diabetic ulcers, it was found that some of the few commercially available products had a substantially debridement effect, others were only granulating and others were just re-epithelializing. Moreover, their positive effects in the beginning were reduced or suffer from some drawbacks, such as poor adhesion or poor absorption, and in some cases preparations acted as an occlusive material, in other producing excessive dehydration of the wound surface and of the wounds secretions, and further the remains of the precipitated product adhered to the wound resulting in scabs.

Specifically, the indicated preparations for the “debridement” of the wound (often improperly called, “healing”, i.e. products generally proposed to accelerate the healing of wounds, regardless of what stage of recovery process that we refer to) had beneficial effects in cleaning the beds of the wounds, but their use beyond this stage did not support the effort of the body to granulate and to fill the ulcer space with a granulation tissue. Some products marked as “granulating”, that is active in stimulating the occurring and the growth of granulation tissue, strongly adhered to the tissues and acted as an occlusive material, and besides being difficult to remove, drying up the wound and produce scabs. Finally, essentially “re-epithelializing” preparations were particularly useful in the last stage of repair of a deep wound with tissue loss (or superficial wounds such as burns of first degree, even after the onset of the treatment), but if applied indiscriminately in the phase that does not match, they had the effect of hampering the processes of wound repair.

### SUMMARY OF THE INVENTION

In the light of the foregoing, the object of the present invention is thus to provide for therapeutic means useful for the topical treatment of wounds having a difficult or slow healing, such as—specifically, open wounds with loss of tissue, and in particular the lacerated-contused wounds and ulcers, including diabetic ulcers, as well as burn wounds.

Starting from the concept of the utility and need to supply a moist medium to wounds, so that any living tissue can grow in the most propitious environment, compositions containing natural active principles have been designed from which, according to the invention, moist medicaments and dressings have been prepared. The latter have a contents of actives é partially different from one another, depending on the stage of development of the healing process in which they are applied.

According to the invention, the preparations had to meet the following requirements:
1. easily absorbed active components, in order to act by absorption and diffusion from the site of application in the wound bed to the surrounding tissues rather than by contact, with controllable systemic repercussions;
2. presence of nutrients which during absorption and assimilation by the neoformed tissues can ensure to the substrates carbohydrates and minerals, proteins, vitamins and fats required for the regenerative functions in the process;
3. arterial vasodilator effect, at least at the level of the lesion and around the same, with the purpose of improving the chaotic circulation which is common in diabetic patients;
4. stimulating effect for the generation of new arterial blood vessels (neoangiogenesis) which, in combination with the vasodilatory and arterial effects, assures an excellent blood supply;
5. an hemorheologic effect that can result in a lower viscosity of the blood, in order to remove the occlusion of thrombosed veins and the subsequent disappearance from the lesion of the blackish lines corresponding to the above veins;
6. antiseptic and antibiotic properties, such as those that are already known in many medicinal plants currently in use.

Bearing in mind these requirements, a specific formulation has been found based on dried and pulverized medicinal plants, suspended in bees' honey, which in combination with each other in definite ratios can be applied as a topical cream product on a wound, both acute and chronic, both superficial and deep, and having a considerable loss of substance, to stimulate and assist the healing process and recovery of injured tissue, and to accelerate the wound healing.

### DETAILED DESCRIPTION OF THE INVENTION

Therefore, the present invention specifically provides a topical composition the treatment of wounds consisting of a mixture of dried and powdered plants suspended in total proportions comprised between 5% and 80% by weight in bees' honey, wherein said plants comprise:

a) plants of the genus Plantago,

b) plants of the genus Solanum, and

c) plants of the genus Kalanchoe.

These three genera of plants, which are individually known in the field of medicinal plants and traditional herbal remedies, have necessarily to be present, all the three of them, in the formulations proposed according to the invention. To these, a fourth plant genus is added, the genus Scoparia, which is included in the formulation of two of the three specific types of preparations proposed according to the invention, namely the formulations having, respectively, activity in stimulating the granulation of the wound (“granulating” preparation), and activity in stimulating re-epithelialization of the same (“epithelializing” preparation). The fourth plant genus (Scoparia) is not included in the third type of preparation, having the activity of debridement (“debriding” preparation).

According to some specific embodiments of the invention, therefore, the plants of said mixture of plants also comprise

d) plants of the genus Scoparia.

The preparations proposed according to the invention, which have been called “Hidyt”, are characterized by being composed exclusively of plants naturally treated and honey, without any artificial additives or preservatives.

More specifically, the plant part of the topical compositions here proposed consists of dried plants of the species Plantago spp., Solanum spp., Kalanchoe spp. and Scoparia spp.

More specifically, the plants of the genus Scoparia are represented preferably by Scoparia dulcis, a species especially widespread in tropical South America, where it is traditionally used by local people for infusions and for external applications, with a variety of indications resulting from tribal or folk medicine, including pain relief, antiseptic and anti-inflammatory properties.

Plants of the genus Plantago (plantain) can be represented, in particular, by Plantago lanceolata or from Plantago major, also used since ancient times as herbal remedies, not only in tropical areas, but also in temperate areas, especially for their antimicrobial, antiseptic and anti-inflammatory properties.

The plants of the genus Solanum are a very wide family, which includes species cultivated for food production, such as tomato (Solanum lycopersicum), potato (S. tuberosum) and eggplant (S. melongena), and numerous and wild species used as herbal remedies or herbal products. Specifically, the compositions according to the invention may contain plants of the species Solanum dulcamara (climbing nightshade), used as a topical herbal remedy for abrasion and against some dermatoses, and Solanum nigrum (black nightshade) also known as S. incertum, also used as a herbal remedy against inflammations and skin diseases.

In the proposed composition according to the invention, plants of the genus Kalanchoe are represented preferably by Kalanchoe pinnata, traditionally used among the people of the Amazon and in many other areas of South America both for systemic use, in infusions, or for external use, even for treatment of wounds, burns, ulcers and insect bites.

The preparations obtained from the vegetable mixtures described, suspended in bees' honey and in which plants Plantago spp., Solanum spp. and Kalanchoe spp. are used, are particularly suitable for the first phase of therapy according to the invention, when an activity of debridement of the wound is specifically requested. In this case, the mixture of dried and pulverized plants contains Plantago spp., Solanum spp. and Kalanchoe spp. preferably in weight ratios between 2:2:3 and 4:2:2. respectively.

In the case where the preparation proposed is to be used in the next phase of the healing process, when an action of stimulation of the wound granulation is primarily requested, the mixture of plants indicated according to the invention contains Scoparia spp., Plantago spp., Solanum spp. and Kalanchoe spp., preferably in weight ratios between 1:1:3:1 and 1:2:4:1.

Finally, for a wound in which the tissue initially missing has been substantially replaced by granulation tissue, and that has to undergo the last part of the proliferative phase, i.e. that of re-epithelialization, the topical composition indicated according to the invention, having re-epithelializing activity, includes a mixture of Scoparia spp., Plantago spp., Solanum spp. and Kalanchoe spp., preferably in respective weight ratios of between 8:4:4:1 and 16:4:4:3.

According to another complementary aspect thereof, the present invention relates to a medicinal preparation containing, as active ingredients, a mixture of dried and pulverized plants suspended in overall ratio between 5% and 80% by weight in bees' honey, which plants include:

a) plants of the genus Plantago,

b) plants of the genus Solanum, and

c) plants of the genus Kalanchoe.

for use as a topical medication. This definition includes the “debridant” version of the preparation of the invention, specifically designed for use in the process of debridement of wounds, wherein the mixture of Plantago, Solanum and Kalanchoe, preferably Plantago spp., Solanum spp. and Kalanchoe spp., is used, preferably in weight ratios of between 2:2:3 and 4:2:2.

According to other embodiments, the invention relates to a preparation containing a mixture of dried and pulverized plants suspended in overall ratios between 5% and 80% in bees' honey, wherein said plants include:

a) plants of the genus Plantago,

b) plants of the genus Solanum,

c) plants of the genus Kalanchoe, and

d) plants of the genus Scoparia

for use as a topical medication. This definition includes, in turn, the “granulating” version of the preparation of the invention, specifically designed for use in the development phase of the granulation tissue, and the “re-epithelializing” version of the same preparation, specifically designed for use in the subsequent stage of re-epithelialization. In the first case the mixture of Scoparia, Plantago, Solanum and Kalanchoe, preferably Scoparia spp., Plantago spp., Solanum spp. and Kalanchoe spp., is used preferably in the weight ratios of between 2:2:3 and 4:2:2, while in the second case the mixture is preferably used in weight ratios of between 8:4:4:1 and 16:4:4:3.

As already noted, the Hidyt creams have been developed and tested in diabetic patients suffering from severe injuries at the level of the lower limbs, such as neuropathic, ischemic, infectious and traumatic type. These are often deep and large wounds, affecting the entire heel or instep, or all of the big toe or all toes, or the entire sole of the foot, and leave exposed the deep bone structure, in many cases with plurimicrobic purulent infections, and with large areas of necrosis which involve the tissues up to the bone. They were also tested in diabetic wounds in different parts of the body as in parts of the buttock, pubic, abdominal and back areas, as well as the neck and hands. In all these cases the results were excellent.

The compositions of the invention were also tested on patients not suffering from diabetes, but which, for some situation, had developed ulcers of different sizes and gravity. Were applied in varicose ulcers, traumatic wounds, pressure sores, burns and even in a case of longstanding facial chloasma. The compositions have also been applied as an intravaginal cream in patients with ulcer of the cervix and in cases of hemorrhoids, and in all applications, the results were excellent.

With specific reference to the requirements 2 and 3 given above (the effect of arterial vasodilation and stimulating neo-angiogenesis), these two properties have been successfully obtained in preparations of the invention, as shown by the deep red color of the lesion and particularly of the granulation tissue, and from the plethoric appearance (i.e. over-irrorated by blood) of all the treated area during therapy. Considering specifically the injuries to the foot, it is noted that once the process of regeneration is ended and the wound is completely closed, the healthy foot is swollen. This phenomenon increases when the patient walks and the inflow of blood to the entire foot is such that the foot becomes swelled and warm and takes on a bluish color. For this reason it is recommended to the patient to stop and raise his/her foot, which is deflated immediately, or alternatively to walk short distances.

The event described remits spontaneously over a period of between one and three months, at which time the newly formed vessels in the repaired wound close or degenerate, by virtue of the fact that it is not needed such a high amount of blood in the injured area. This contribution is very useful during the period of active granulation and repair of tissues, but it must be reduced gradually in the later stages of healing.

Concerning the requirement no. 5 mentioned above (hemorheologic effect that will result in a reduction of blood viscosity), results have been obtained from the use of the invention preparations Hidyt, that can be attributed to an antiplatelet, or fibrinolytic, or antithrombotic or thrombolytic activity; or to the presence of any of these activities, the interaction of some of them with one another, or even all.

During the initial preparation trial, the achievement of these results has been demonstrated in some cases with the presence of considerable hemorrhages, which were revealed by the stains on the sheets of the patient. To avoid undesired or excessive effects, the concentrations of the components initially set were suitably reduced, but in any case possible bleeding effects of the proposed preparations are controlled with the intravenous administration of vitamin K, to stop the hemorrhage. In examined patients the value of the clotting time, represented by PT (prothrombin time) and PTT (partial thromboplastin time) are prolonged. At least this fact shows that the components of the topical preparation proposed, as regards the activity hemorheologic, have a systemic action.

The cases cited allowed to establish, through experiments conducted on each of these plants taken separately, the effects attributable to each of them in terms of reducing the viscosity of the blood are not sufficient to justify the effect achieved by the composition of the whole. Therefore it was deduced that in this case, as in the case of other properties, the fact of mixing the various active ingredients in certain proportions to each other produces a true synergism of reinforcement, which results in an unexpected healing effect.

With reference to the requirement no. 6, the preparations of the invention have antibiotic properties, both in consequence of the properties of the honey that forms the vehicle, or for the known antibiotic properties of some of the plants of the composition.

Purulent and short-changing infected wounds were treated with preparations of the invention, in septic patients and in a serious, decompensated and anemic state, in which previously undertaken antibiotic therapy had failed; or long-standing wounds, clean and having in incomplete granulating, or completely granulated or in an incipient phase of re-epithelialization. Finally, wounds have been treated in which for some reason the normal evolution of the healing was interrupted at any stage of the cure.

The preparations proposed and the treatment techniques thereof have been successfully tested on lesions evolving one, three, eight months, as well as lesions of long standing, who had also originated forty years before, which highlighted old tissues prepared in a disorderly manner, united with one another in different phases within the same wound, granulation tissue, edge regions in the incipient phase of re-epithelialization, areas with large amounts of necrotic tissue and debris, secretions. The interesting aspect is that in these wounds, already two or three days from the beginning of treatment with the preparations of the invention, the improvements were manifested in a surprising way, up to reach in a few weeks at a total cure. From this point, it follows that the proposed preparations have the ability to activate cell regeneration of blood vessels, granulation tissues and skin in an orderly and harmonious manner, regardless of environmental conditions of the lesion and of the reparative phase where the wound was maintained.

At least during the granulation stage the plants in the study inderwent to experimental verification in an individual manner. In this way it has been found that the fact of mixing the plants between them in certain fixed raios and, especially, of using different raios depending on the reparative phase in which the healing process is, gives results surprisingly better than as might be expected based simply on an “addition” of their property.

### Properties of Hidyt Preparations

Anti-inflammatory—The topical preparations of the invention have potent anti-inflammatory action which is visible not only in the wound bed but also in the skin around the lesion, changing the erythema to normal, cyanosis and edema in the initial wound bed, the red wine of the traumatic and infected wounds gradually changes to bright red. For this reason, once the topical treatment begins to be applied, oral anti-inflammatory are not required. This fact is of great benefit, since it avoids the possible gastrointestinal, renal and other nature consequences, resulting from the indiscriminate use of anti-inflammatory drugs (NSAIDs).

Stimulating the secretion—humectant—The main mechanism by which the preparations of the invention achieve the anti-inflammatory and deflation effects may be related to their wetting properties and stimulating the secretion. This property, properly regulated, softens the tissues and facilitates the removal of debris from the wound. This property is of particular importance because, while in other cases and with other techniques substances must be used such as to provide a humid environment to the wound by the application of dressings, in this case the wound itself, by induction of the preparation, is kept moist while it deflates, dehydrating the inflamed tissues up to useful limits. This property has been properly modulated, according to the invention, without having adverse effects on the benefits partial and final.

Stimulating the debridement—the properties of the preparations of the invention of stimulating debridement is the result of the chemical action of one or more of its components on the tendon and connective tissue, on the purulent material, the necrotic tissue and debris of the local wound.

The different plant components of the preparation were tested separately on bovine muscle and connective tissue for eight days, and the results were not very convincing. However, two of the plant components of the proposed preparation plant showed protease activity by approximately 33%. Mixing the products between them, instead, their effects have proved to be significant, probably because of a synergism between them, or between those and the enzymes or other products secreted from the wound itself.

As a result of this activity, we observe that day after day, the fibrous tissue that lies in the wound is losing grip and consistency, they can be removed easily from the wound, and in some cases remains adherent to the patch when you run the dressing at home, as is usually reported by patients themselves.

Thrombolytic—In the bed of varicose ulcers is common to see the tortuous routes of azure thrombosed veins in the wound bed and around the leg. For this reason the use of anticoagulant is indicated as part of treatment of these ulcers.

In this type of injury as early as the first week of applying the “debridant” cream thrombosed veins disappear completely without leaving the slightest trace of their existence, and this benefit is maintained through the successive application of other types of preparation according to the invention.

Stimulating the granulation—The appearance of the granulation tissue occurs regardless of the fact that the cleaning and the debridement of the wound are completed and it has the same origin as a substrate the same healthy connective tissue of the surface layers, the arteries and veins that remain exposed, the muscle fibers and bone marrow when it is exposed.

The stimulatory granulation activity fills all the gaps and recesses that can be left as a result of massive tissue destruction caused by infection, even those of the anatomy of body organs such as the foot and hand. This is a tissue of new formation, of exuberant red light, grainy, which bleeds easily, covering the whole extent of the wound and involving and surrounding tendons, arteries, veins, bones and nerves, without cutting off functionality. After filling the deep voids, it grows toward the surface until above the level of the skin around the wound edges.

The granulating activity is the most important property of the preparations proposed according to the present invention. It is the result of all the desired properties and produced in the Hidyt preparations, where are required properties of vasodilation and fluidizing in the blood, neoformation of blood mainly arterial vessels, additional nutrients, anti-inflammatory and moisturizing properties and the antibiotic effect.

The initial development of this granulation tissue is exuberant, with large granules that day by day grow excessively. However, at the beginning of the activity of re-epithelialization and of the change of the type of product according to the invention, the granulating activity is slowed and the tissue that was grainy becomes smooth, and the part that initially exceeded the level of the skin surrounding the lesion planes at the same level of healthy skin in perfect harmony.

It has also been tried on several occasions that if the treatment with the preparations of the invention is interrupted at this stage in favor of another epithelializing preparation, for example, commercial, the healing process slows down macroscopically and the granulation tissue is back smooth. After the restarting of the application of the preparations according to the invention the same regenerating power of the large granules comes back clearly.

Reepithelializing—One of the most difficult challenges to achieve in a long-standing injury is undoubtedly the beginnings of a re-epithelialization.

According to some theories, this takes place through the proliferation of the epithelium of the edges of the wound and the subsequent slippage of new epithelial cells from the edges, in the centered towards form within the wound bed.

In the application of the preparation according to the invention, in its “reepithelializing” form, it was found several times, unexpectedly, the presence of nuclei of re-epithelialization in the center of the lesion, or at some other point in the wound bed, separated from the active edge. In some cases these nuclei of re-epithelialization appear prior to the re-epithelialization which starts from the edges.

The new skin is the same as the original skin, and gradually resumed normal color, so that practically no scar remains. The skin replenished, also recovers normal sensitivity.

### Other Properties'

It is common that the product of the normal secretion of wounds, over the granulation tissue and between the pores and the spaces which are among the granules, is deposited as a transparent gelatinous material, whose consistency and adherence to the granulation tissue increases with time. This is the fibrin, which dries and gives rise to the crust that covers the aged wounds. In many cases between the surface of granulation tissue and this crust is formed a space occupied by pus.

With the use of preparations according to the invention the production of fibrin is minimal, if not practically null, of little consistence and of little adherence to the granulation tissue.

This fact is particularly important, because if the wound must be preserved free from fibrin the frequency of medical examinations required for her recovery-region and their total number is less.

### Method of Application

The use and application of the three topical preparations of the invention must be done in sequence: first, the product for the “debridant” action is applied, followed by granulating and finally the epithelializing preparation.

The “debridant” preparation must be applied after a control of the infection, and the application continues until all the fibrous tissue and debris were removed.

The preparation for granulating action is to be applied when the wound is completely clean, and its application continues until the granulation tissue has filled all the spaces, tunnels, caves and crevices formed by the infection, and has completely covered the bed of the wound so as to leave no difference in level between the bed itself and the normal skin around the lesion.

The epithelializing preparation is applied from this moment, and since his aim is to cover all the uncovered area of skin, it is applied until it is necessary.

It was noted that during the re-epithelialization stage, in the skin around the new active edge very tight scabs are formed, which are better to remove. This fact persists until the wound has completely closed as a crust having color of coffee in the center of the scar, but eventually it disappears, and the skin takes the normal color and texture without signs of the former presence of a wound so bloody.

Not all of the wounds will require that the treatment is started with the preparation for the debridement: the type of preparation according to the invention which is the most appropriate to be applied at the beginning of the treatment will depend on the conditions of the lesion. For example, if at the first visit the wound to be treated is clean and without purulent or fibrous material, or debris, it has to start with the granulating type. If it is a superficial wound, as in the case of a first-degree burn, or the wound has completely granulated and with good vascularization, in this case we may begin with the re-epithelializing type.

In several cases the granulating preparation already operates the complete closure of the lesion, or the application period of the next re-epithelializing type is shorter than normal. This occurs because the three varieties of the preparation according to the invention, each one being not versatile, as they perform their main functions, they also complete the previous phase, or prepare and promote the conditions for the preparation of the next step so as the latter may carry out its main task. Thus the granulating preparation has as its main function in stimulating the granulation to fill the spaces of the connective tissue, but also has some debridement activity allowing the provision of a limited emollient properties to easily remove any debris or fibrin residue that may still occur. At the same time, has a mild stimulating effect of re-epithelialization, which is demonstrated by the color change of the edges, which begin to activate to revert to a rosy color, while the granulating activity continues.

The indications with respect to the frequency of application of the preparations according to the invention are preferably the following: the debridement variety applies every day, once a day, up to achieve the softening and the elimination of all the devitalized material. Normally its use is required for no longer than five days.

The variety stimulating the granulation is applied on alternate days once a day, and the epithelializing variety is applied once every two or three days. However, also these two varieties may be applied daily.

Overall, the results obtained with the clinical trial are surprisingly: a maximum of three months cases were retrieved that were considered for lost, and where the diagnosis included the amputation.

### DESCRIPTION OF THE FIGURES

The clinical results obtained with the application of the preparations according to the invention are illustrated but not limited by the experimental cases reported in the following, as well as in the attached photographic documentation, which shows the evolution of an exemplary clinical case treated with the compositions and the therapeutic procedure of the invention.

The attached figures show dated photographs in succession of the foot of a patient with a traumatic injury caused by an animal leg crush, with infection and amputation of the second and third toe.

FIGS. 1a and 1b show the situation after the initial amputation and the consequent severely inflamed status;

FIGS. 1c and 1d show the situation about a month later, when the inflammation has been controlled and in its place a well demarcated area of necrosis is visible, which affects the big toe and fourth toe;

FIG. 1e shows the surgical wound on the day of the amputation of the two necrotic fingers, the day when therapy begins with the topical preparations according to the invention;

FIGS. 1f and 1g show two following stages of the therapy according to the invention, in which are evident, respectively, the still bleeding ends of the two recently amputated fingers and the formation of abundant granulation tissue;

FIGS. 1h and 1i show other two following stages of the healing in which the granulation tissue has become more compact and re-epithelialization is the main event.

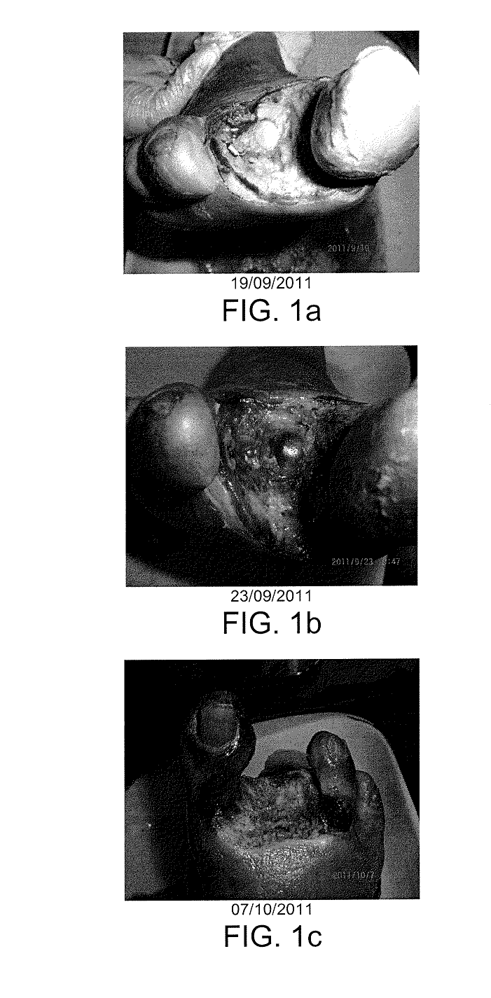

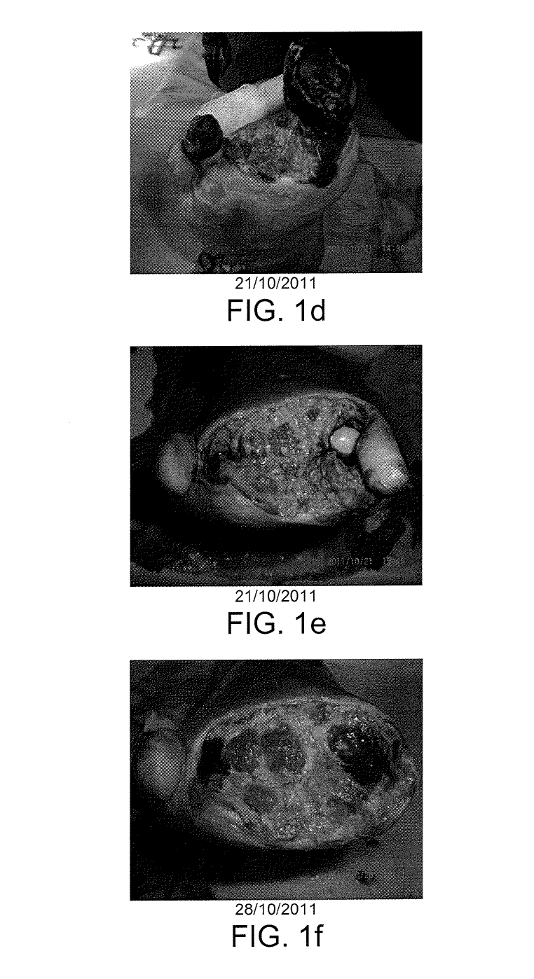

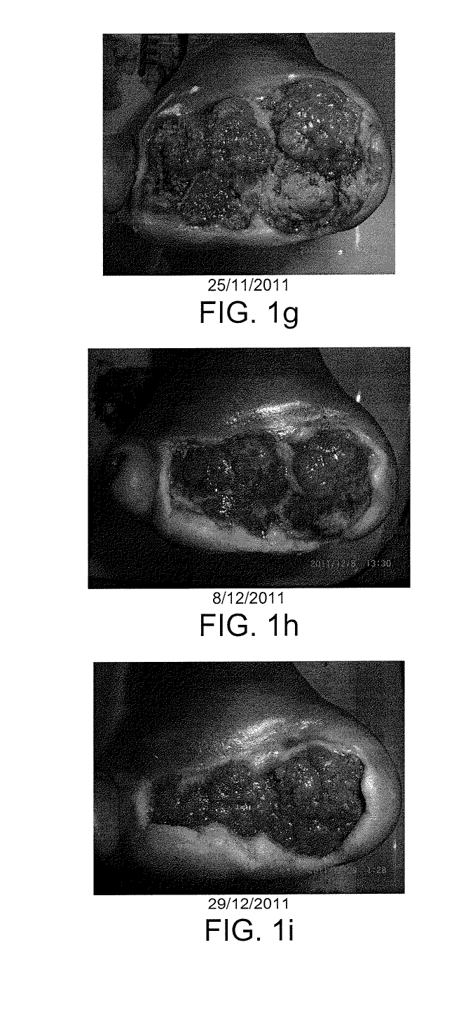

Clinical Trials on Patients with Wounds Treated with Typical Hidyt Preparations

### First Case—Burn Wound Infection Complicated by Hot Substance

Diabetic female patient since a long time, 48 years of age, which two years before has undergone the amputation of the external malleolus of the left foot showing fistulas up to the tibio-astragaline joint. Despite a conventional treatment by the same team of doctors who had performed the amputation, she did not respond satisfactorily. The wound sometimes secretes serous fluid and sometimes pus, or gets closed.

At the time of the first visit the patient had fever, the wound was in the process of secretion, surrounded by an erythematous bright halo; family members said that it should be due to a first degree burn produced by a warm compress on the site. Incision is made, which drained a lot of yellow-blackish pus, foul-smelling.

The case was complicated by the fact that two superimposed layers of wounds had to be treated, the first deeper that reaches the articulation itself, and the other superficial, which widens the first. It has been defined the entire surface of the wounds, leaving exposed the subcutaneous connective tissue, tendons and ligaments of the joint. Once controlled the infection, we applied the topical preparation of the invention having debridement activity for three consecutive days.

After a month of treatment, the wound appeared in clear granulation. The subcutaneous connective tissue, tendons and ligaments remained viable despite the infection, but during the granulation process, obtained by the application of the granulating preparation of the invention, tended to be destroyed.

At nine weeks, the granulating activity filled the whole thickness, but the re-epithelialization was active, covering the front part of the wound. The subcutaneous connective tissue and fat were completely lost.

At the tenth week granulation and epithelialization were active. A small residue of the tendon remained, and a perilesional edema was noted, which is not synonymous with inflammation, but a sign of a great vascularization.

At the 14th week, the granulation was completed, and the granulation tissue that was crude became smooth. There remain residues of connective tissue or tendon, and the re-epithelialization covering the exposed surface continued. The appearance of the foot was pink, bloated.

Four months after, the new skin had nearly covered the whole bed of the wound, but the bottom over the new skin, near the center yet discovered the presence of a crust is noted, which came off easily leaving new pink skin. A week later the wound was totally re-epithelialized.

Despite the loss of the tendon, the patient could walk without any problem. We had to put a prosthetic to the amputated leg.

Several months later she returned to a medical examination and was found in perfect condition. The skin had the same characteristics of normal surrounding skin.

### Second Case—Chronic Wounds, Diabetic Male Patient

Wound aged six months of development, which was treated with a drying powder, located at the root of the left foot toe nail. The wound appeared covered with fibrous connective yellowish tissue covered with white powder, with which he had been treated, the granulation tissue below appeared smooth and rosy.

A surgical debridement was performed to remove the macerated fibrous tissue and the treatments according to the invention were started with the preparation for granulating activity. After one week of treatment, the wound has been granulated turning the smooth granulation into a granulation with large granules and covering the entire wound bed of the alive tissue. The new skin had covered about 40% of the wound surface.

After two weeks, the granulation and epithelialization were active.

After 26 days of treatment, the skin had covered 90% of the surface of the wound, and a week later the wound was completely closed.

Six weeks later the patient returned for a medical examination, the wound was covered with skin which persisted over the crust in the center of the original wound.

### Third Case—Varicose Wound to the Instep and External Perimalleolar Area

Non-diabetic female patient, suffering from varicose veins in the left leg, which spontaneously developed a wound that in a few weeks became very large.

The patient was hospitalized for eight months in vascular surgery, and during this period various techniques and different types of creams of the best internationally known were applied, without achieving the desired results.

The patient showed a lot of pain, because of which she adopted analgesic positions walking. There were two related injuries, one on the instep and the other at the top of the lateral malleolus. By the dark brown crusty appearance it was noted that the wounds were aged and crusted allowing to see in transparency the muscle plan and tendons in one case and the lower end of the tibia in the other case. The edges of the wounds were necrotic.

After just three days of application of preparation with debriding activity according the invention a change in the appearance of the tissues was noted: tissues initially necrotic dry and very adherent to the deep layers at the time to remove the gauze before cleaning, were moist and hydrated and softened, and the living tissue appeared red for the action of the debridant preparation. Following the application of the product, that exerts a destructive action on tissues already necrotic and on those in the process of necrosis, a “isthmus” of skin that initially divided the wound into two parts, degenerates and is eliminated by the treatment. Once the cleaning of the wound was obtained the benefit of the preparation was clear.

After ten days of treatment and seven days of application of the granulating version of the preparation of the invention, the granulation was in full activity. The particular adhesion of the residues of plant material to the tendons was noted, and the latter began to appear devitalized and wearied. The wound was completely clean and full of granulation. The tibia was almost all covered by a neoformation tissue.

After At two weeks of application of the granulating preparation bleeding began to appear, which continued throughout the week, as was evidenced by the ecchymotic color of the fibrin impregnated by extravasated blood. At the time of removing the fibrin bleeding was widespread, tendons continued to waste and the tibia appeared completely covered by granulation tissue.

After three and a half weeks of treatment and three weeks after the application of the granulating preparation the granulation phase was completed and the re-epithelialization phase was in full development. The activity of collagenase was such that it was sufficient to take the tendons humidified with the gripper to clean the wound, despite the fact that the main function of the applied preparation was the regeneration. The granulation tissue appears thick, and the re-epithelialization was active since a bit of time, and the edges of the wound bed had been covered by the new skin.

Two and a half months after, the process of re-epithelialization progressed slowly because of the inconstancy of the patient and the bad treatment of the wound, as a lack of rest and a too tight bandage. There remained very little portions of the tendons, the granulation tissue was smooth and the new skin covered it by the edges. The re-epithelialization was active, the texture and color of the skin tended to approach the normal skin color.

It is important to clarify that, in this patient, the results, though being still exceptional, were not able to get the completion because the patient has not complied with the treatment program, and several times she gave it up, and then returned to follow it when the wound was getting worse. She also suffered a series of incidents during the period of treatment, including a burn with hot water and one with an acidic substance that, falling over the wound, caused a regression so severe that it hurted all the results hitherto achieved.

Following each of these episodes it was possible to recover the injury-treating it with the topical preparations of the invention. The last time I visited the patient, the wound was regenerated up to 80%.

### Fourth Case—Critical Ischemia Wound of Lower Limb, Diabetic Male Patient

Diabetic patient, 48 years of age, pale, anemic, fever, blood glucose 320 mg. Diagnosed with critical ischemia of the lower left limb, which is corroborated by arteriography showing the clogging of the deep femoral artery in the medial third and downward obstructions total and partial across the path of the terminal branches of the same, below the popliteal branches, and a development of poor arterial collateral cycle.

The decision of the doctors in general and vascular surgery was to operate an amputation of the leg until the knee cap, secondary to the lack of response at the surgical transmetatarsian amputation of the big toe, despite treatment with systemic anticoagulation and topical preparations in cream, and special care.

For the opposition of the patient to amputation, as a last tentative, for 15 days the patient was subjected to daily sessions of rectal ozone therapy. In the absence of evident signs of improvement in the wound that would give hope to have improved perfusion in order to ensure the tissue regeneration and healing of the wound, a treatment with the preparations according to the invention was initiated.

At the first consulting visit the foot appeared pale and emaciate, the wound base rosy, covered by abundant connective tissue devitalized. The second finger, necrotic and hardened, was amputee and the wound was debrided by surgery. The bleeding was moderate, and the granulating preparation Hidyt to 50% has been applied.

At the second consulting visit, three days after starting the treatment, the wound was already red, covered with blood clots, the bleeding persisted for two weeks, and this time it was significant and detectable by the sheet. The patient reported that the bleeding occurred mostly when he put the leg to the ground. The granulation tissue was red, with granules moderately thick, and the skin of the edges showed signs of reactivation. At one month of treatment, the foot was not inflamed but plethoric, with no pulse at the instep, or tibial rear. The wound was red and granulating showing active re-epithelialization from the top and from the outer edges.

In the following week the granulation tissue was thick and re-epithelialization was activated starting from the edges.

The therapy was continued every other day for seven months, without any gaps, and until the last day bleeding from the undiscovered persisted and the foot was still bloated, hot and pink, becoming dark red and more bloated when it was lowered.

### Fifth Case—Infected Diabetic Foot

The diabetic pathological event had affected the big toe finger, the area of the instep and went up over the outer ankle bone and under the anterior part of the plant. After a month of conventional treatment and without apparent cause it occurs a injury in the toe finger. Despite treatment evolution was chaotic.

At the first examination the patient had fever, he was cold, with all the left leg swollen and inflamed, with blisters and shiny skin, plump up to the medium third of the leg. The finger toe slightly cyanotic with plaques of necrosic skin in the dorsal side and more signs of necrosis of the finger that made it no longer viable. From the rear, behind the hanging of the fingers a crusted lesion was present closed, which once engraved drained abundant blackish stinking pus, typical of an anaerobic infection. The skin was pale, flaccid with changes on the inside of the foot.

The day following the incision surgery to drain pus, skin color and edema were improving, tendons and fascia remained exposed. At the fifth and sixth day of topical antibiotic treatment all the broken skin and the subcutaneous connective tissue devitalized were dried, the color of the skin around the lesion was tending to normal, and wrinkles showed that inflammation was under control. After controlling the primary infection appeared an infection by Pseudomonas, characterized by the typical greenish color and smell of rotting vegetables. This situation forced to postpone the application of the preparations of the invention (Hidyt) to further eight days. The same infection appeared again 15 days after the starting of the application of the Hidyt debridement preparation.

Although it had not yet begun the application of the Hidyt granulating preparation, a granulation tissue below the fibrous tissue begun to be appreciate.

After three weeks of treatment, the big toe was amputee and the most distal surface of the finger metatarsal was cut to reach the trabecular bone of the epiphysis, where is located the hematopoietic bone marrow. From this tissue, at the third day of treatment with the preparation of the invention, also begun the generation of a granulation tissue, which often grew with a rapidity never seen before. The granulation tissue is often exuberant, red and very bleeding.

Six weeks of treatment and three and a half weeks from the application of the granulating Hidyt preparation variety in studio, the wound presented a thick granulation tissue, as to be seen as an exuberant malignant tumor excrescence. Between the grains a more intense red color was appreciated due to hemorrhage, which in this patient, as in other, appeared to be significant. The Incipient re-epithelialization appeared with pink edges.

After eighth weeks of treatment and at the fifth week of application of the granulating preparation, the granulation phase was complete, and the top phase was the re-epithelialization one. At this time there is an interphase in which coexist the granulation phase and re-epithelialization phase and granulation tissue easily exceeds the level of normal skin.

As already noted, during the granulation process the tendons, fascia and ligaments were lost and decomposed.

At the sixth week of application of the preparation the re-epithelialization was active, the granules of the granulation tissue were more smooth and the new skin covered the wound from the outer edges. Two weeks later the foot was totally healthy, as much the wound on the neck of the foot and its extension towards the upper part of the lateral malleolus, as the plantar wound and the stump of the finger toe.

The present invention has been disclosed with particular reference to some specific embodiments thereof, but it should be understood that modifications and changes may be made by the persons skilled in the art without de-parting from the scope of the invention as defined in the appended claims.

1. A topical composition for the treatment of wounds consisting of a mixture of dried and powdered plants suspended in total proportions comprised between 5% and 80% by weight in bees' honey, wherein said plants comprise:
a) plants of the genus Plantago,
b) plants of the genus Solanum, and
c) plants of the genus Kalanchoe.

2. A topical composition according to claim 1, wherein the said plants include, in addition
d) plants of the genus Scoparia.

3. A topical composition according to claim 2, wherein the said plants a), b), c) and d) respectively consist of plants of the species Plantago spp., Solanum spp., Kalanchoe spp. and Scoparia spp.

4. A topical composition according to claim 3, wherein the said mixture of dried and powdered plants is suspended in total proportions comprised between 20% and 60% by weight in bees' honey.

5. A topical composition according to claim 3, wherein the said Plantago plants are Plantago lanceolata or Plantago major.

6. A topical composition according to claim 3, wherein the said Solanum plants are Solanum nigrum or Solanum dulcamara.

7. A topical composition according to claim 3, wherein the said Kalanchoe plants are Kalanchoe pinnata.

8. A topical composition according to claim 3, wherein the said Scoparia plants are Scoparia dulcis.

9. A topical composition for the treatment of wounds according to claim 1 having debridement activity, wherein said mixture of plants contains Plantago spp., Solanum spp. and Kalanchoe spp. in weight ratios comprised between 2:2:3 and 4:2:2.

10. A topical composition for the treatment of wounds according to claim 2 having granulation stimulating activity, wherein said mixture of plants contains Scoparia spp., Plantago spp., Solanum spp. and Kalanchoe spp., in weight ratios comprised between 1:1:3:1 and 1:2:4:1.

11. A topical composition for the treatment of wounds according to claim 2 having reepithelialization stimulating activity, wherein said mixture of plants contains Scoparia spp., Plantago spp., Solanum spp. and Kalanchoe spp. in weight ratios comprised between 8:4:4:1 and 16:4:4:3.

12. A medicinal preparation containing, as active ingredients, a mixture of dried and powdered plants suspended in bees' honey in total proportions comprised between 5% and 80% by weight, wherein the said plants comprise:
a) plants of the genus Plantago,
b) plants of the genus Solanum, and
c) plants of the genus Kalanchoe.
for use as a topical medicament.

13. A medicinal preparation according to claim 12, for use as a medicament for the debridement in the treatment of wounds.

14. A medicinal preparation according to claim 13, wherein the said mixture of plants contains Plantago spp., Solanum spp. and Kalanchoe spp., in weight ratios comprised between 2:2:3 and 4:2:2.

15. A medicinal preparation containing, as active ingredients, a mixture of dried and powdered plants suspended in bees' honey in total proportions comprised between 5% and 80% by weight, wherein the said plants comprise:
a) plants of the genus Plantago,
b) plants of the genus Solanum,
c) plants of the genus Kalanchoe, and
d) plants of the genus Scoparia
for use as a topical medicament.

16. A medicinal preparation according to claim 15 for use as a medicament stimulating the granulation in the treatment of wounds.

17. A medicinal preparation according to claim 16, wherein the said mixture of plants contains Scoparia spp., Plantago spp., Solanum spp. and Kalanchoe spp., in weight ratios comprised between 1:1:3:1 and 1:2:4:1.

18. A medicinal preparation according to claim 15, for use as a medicament stimulating the reepithelialization in the treatment of wounds.

19. A medicinal preparation according to claim 18, wherein di the said mixture of plants contains Scoparia spp., Plantago spp., Solanum spp. and Kalanchoe spp. in weight ratios comprised between 8:4:4:1 and 16:4:4:3.
